(19) United States
(12) Patent Application Publication
(10) Pub. No.: US 2005/0095699 A1
(43) Pub. Date: May 5, 2005

(54) Functioning substrate with a group of columnar micro pillars and its manufacturing method

(76) Inventors: Akihiro Miyauchi, Hitachi (JP); Kosuke Kuwabara, Hitachi (JP); Masahiko Ogino, Hitachi (JP); Hiroshi Yoshida, Mito (JP); Takashi Ando, Hitachi (JP)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/985,972

(22) Filed: Nov. 12, 2004

Related U.S. Application Data
(63) Continuation of application No. 10/695,939, filed on Oct. 30, 2003.

(30) Foreign Application Priority Data
Oct. 30, 2002 (JP) 2002-315214
Oct. 30, 2002 (JP) 2002-315217

(51) Int. Cl. C12M 1/14; C12M 3/04

(52) U.S. Cl. 435/299.1

(57) ABSTRACT

A functioning substrate with a group of columnar micro pillars characterized in that a first matrix of organic polymer and a group of columnar micro pillars of organic polymer extending from this matrix are provided. This group of columnar micro pillars has an equivalent diameter of 10 nm through 500 μm and a height of 50 nm through 5000 μm; and the ratio (H/D) of the equivalent diameter (D) to the height (H) of the group of columnar micro pillars is 4 or more.

### CROSS REFERENCE TO RELATED APPLICATION

This application is a continuation of application Ser. No. 10/695,939, filed Oct. 30, 2003, the contents of which are incorporated hereby by reference.

### FIELD OF THE INVENTION

The present invention relates to a functioning substrate or a functional element equipped with a micro pillar group of organic polymer and to its manufacturing method. It also relates to a micro biochip or an optical device using the same.

### BACKGROUND OF THE INVENTION

Non-patent document 1 (Applied Physics, Vol. 71 (10), pp. 1251-1255, 2002) discloses a technique of forming a nano-silicon pillar group using a metallic cluster such as iron, gold and silver as the self-forming nucleus of a plasma etching mask, and creating a photonic crystal (optical device) by removing a specific cylindrical row. The nano-silicon pillar formed on the silicon substrate has a diameter of 50 nm and a height of 1 μm, with a cluster cycle (pitch) of 500 nm.

The light having a waveform of about twice the cycle of the nano-pillar cannot pass between the pillars due to a photonic band gap, but the portion with one row of pillars removed (linear defect) allows light to pass through. Thus, the incoming light travels through the aforementioned linear defect. Combination of optical signals (ADD), decomposition (DROP) or change of traveling direction in a very narrow space is permitted by proper layout of this linear defect. Use of grating method is preferred when the signal is subjected to DROP. Accordingly, formation of the aforementioned linear detect provides an extremely small optical wave-guide.

Non-patent document 2 (Journal Vacuum Science and Technology B. Vol. 17 (6), pp. 3197-3202, 1999) discloses a technique of forming a resin-made micro pillar. The surface of a silicon substrate is coated with a PMMA (polymethyl methacrylate) film having a molecular weight of 2,000 and a weight of 95 nm. The mask of a silicon substrate is placed on the PMMA film through a spacer, where the distance between the PMMA film and mask is 0.3 μm. Then a sample is heated at a temperature of 130 degrees Celsius for 5 through 80 minutes. When it has been heated, micro pillars are formed on the PMMA film. The mask and spacer are removed from the sample in the final phase. The diameter of the micro pillar is several microns, and the height is described to be 0.28 through 0.43 μm although the spacer determines this.

### DESCRIPTION OF THE INVENTION

The technique given in the aforementioned Non-document 1 uses an etching method to form a micro pillar group on the order of nanometers. The material of the nano-pillar must be removed as volatile gas through reaction with etching gas, and is restricted to such inorganic materials as metal, silicon oxide (SiO₂) and silicon nitride (Si₃N₄). The production method must be restricted to such a dry etching method including ECR plasma etching.

According to the above-mentioned documents, the diameter of the obtained nano-pillar is 15 through 20 nm, independently of cluster size. (see FIG. 5 of the aforementioned non-patent document 1). This means that the wavelength of the light used in the optical device obtained according to this method is restricted to a particular scope.

Of the technique described above, the one given in non-patent document 2 does not use the dry etching method to form the PMMA micro pillar. This allows the micro pillars of organic material (PMMA) to be formed. However, self-organizing of the PMMA film provides a driving force in the formation of the micro pillar. For this resin, it has been difficult to control the position, diameter and height of the micro pillar freely.

### SUMMARY OF THE INVENTION

The present invention provides a functioning substrate or functional element equipped with a group of columnar micro pillars of organic material, characterized by easy control of the micro pillar group dimensions and aspect ratio. The present invention also provides a group of columnar micro pillars according to the handy and less expensive method of manufacturing a functioning substrate or functional element.

The functioning substrate or functioning element equipped with the group of micro pillars of the present invention contains a first matrix or supporting member of organic polymer and a group of columnar micro pillars of organic material extending from this matrix. The micro pillars have an equivalent diameter of 10 nm through 500 nm and a height of 50 nm through 5000 μm. The present invention is particularly suited for use in the functioning substrate having a micro pillar group on the order of nanometers. Accordingly, the aforementioned micro pillar is preferred to have an equivalent diameter of 50 nm or more but less than 1 μm with a height 100 nm or more but not more than 10 μm. It should be noted that the functioning substrate or functional element of the present invention includes a film and sheet.

In the specification, the word “organic polymer” means not only pure organic polymers, but also organic polymer materials containing inorganic or organic fillers or chemically modified materials, unless otherwise specified.

### BRIEF DESCRIPTION OF DRAWINGS

FIG. 1 is a perspective view representing a functioning substrate created in an embodiment;

FIG. 2 is a side view representing the structure of a functioning substrate created in an embodiment;

FIG. 3 is a side view representing the example of the structure of a micro pillar created in an embodiment;

FIG. 4 is a flowchart representing the functioning substrate manufacturing process of the present invention;

FIG. 5 is a Schematic plan representing the configuration of an optical circuit of an embodiment of the present invention;

FIG. 6 is a schematic plan representing the structure of an optical wave-guide oscillation section in FIG. 5;

FIG. 7 is a perspective exploded view representing the configuration of a micro biochip of the present invention;

FIG. 8 is a perspective exploded view representing the configuration of a molecular filter given in FIG. 7;

FIG. 9 is a plan showing the structure of a cell culture sheet of the present invention;

FIG. 10 is a side view showing the culture sheet for cell culture given in FIG. 9;

FIG. 11 is a side view showing the configuration and operation of the water- and oil-repellent sheet of the present invention;

FIG. 12 is a side view representing the operation of a non-colored coloring sheet of the present invention;

FIG. 13 is a schematic side view representing the operation of an anti-reflective layer of the present invention;

FIG. 14 is a schematic side view representing the operation of an anti-reflective layer according to another example of the present invention;

FIG. 15 is a schematic side view representing the operation of an anti-reflective layer according to a further example of the present invention;

FIG. 16 is a flowchart showing the process for forming a nickel thin film layer on the surface of a columnar micro pillar according to the present invention; and

FIG. 17 is a flow chart of manufacturing the functioning substrate according to another embodiment.

### DETAILED DESCRIPTION OF THE INVENTION

It is preferred that the ratio of equivalent diameter (D) to the height (H) of the micro pillar to (H/D, aspect ratio) be 4 or more. This is because the aspect ratio of over a certain magnitude allows desired designing of a functioning substrate or functional element, leading to a wider variety of applications.

In the present invention, the equivalent diameter of the columnar micro pillar means the equivalent diameter at a position intermediate height of the micro pillars. Use of the term “equivalent diameter” is attributable to the fact that the cross section of a micro pillar is not always circular; it may be elliptical, polygonal or unsymmetrical. To include all these shapes, the term “equivalent diameter” is used in the present invention. The aspect ratio (H/D) is preferred to be 4 or more, and is more preferred to be 8 through 30. For the resin of structural strength, however, it is preferred to be 100 or less.

To manufacture the film or substrate equipped with a group of micro pillars of the present invention, a micro mold (a precision mold) with a concave portion (hereinafter referred to as “pit”) having specific arrangements formed thereon is pressed against the thin film made of thermoplastic resin or uncured thermosetting resin, thereby forming a pattern in conform to the aforementioned group of pits of the mold.

The aforementioned mold is made of silicon, quartz, for example. When this mold is separated from the thin film, the thermoplastic resin or uncured thermosetting resin is stretched to form a desired group of micro pillars. Especially, the height of the micro pillars can be adjusted by the aspect ratio of the irregularities (pits), and the position and opening area of the concave portion formed on the mold can adjust the position and bottom area of the micro pillar. This manufacturing method will be described later.

The aforementioned group of micro pillars is preferred to be of self-supporting type. It is also preferred to be capable of becoming independent of the first matrix. Further, it will be good practice to provide chemical modification to the tip end or all surfaces of each of the micro pillars, for example, by providing chemical plating (electroless plating), thereby controlling the reflection factor on the side of the micro pillars.

In the micro pillars constituting the group of columnar micro pillars, the equivalent diameter of the bottom surface is slightly greater than that of the tip end. This is advantageous in ensuring the independence and self-supporting property of the resin-made micro pillars. The micro pillars have a portion tapering from the root in contact with the first matrix toward the tip end. It is also preferred that the group of columnar micro pillars and first matrix be made of the same material. It is essential that the micro pillars and the matrix connected thereto are designed in an integral structure.

The group of the micro pillars of the present invention can be configured in such a structure that the micro pillars are closely packed. This makes it possible to configure such an arrangement that individual micro pillars are less likely to be crushed or to be peeled off from the matrix or the supporting member.

For the process of producing the aforementioned micro pillars, the organic material can be a thermoplastic high molecular material. Or it can be thermosetting molecular material. For example, the main component of the group of columnar micro pillars can be a substance selected from cyclo-olefin polymer, polymethyl methacrylate, polystyrene, polycarbonate, polyethylene terephthalate (PET), polylactic acid and polypropylene. Alternatively, it can be a photo-cured substance obtained by adding a photosensitive material to these materials. Further, it is also possible to add an oxidant inhibitor or flame retardant to these materials.

When the aforementioned mold is pressed against the thin film of thermoplastic resin and is separated, the resin pressed into the pit is stretched to form a micro pillar group that has the equivalent diameter slightly smaller than the inner diameter of the pit but has the length greater than the pit depth. The equivalent diameter and length of the micro pillar depend on the type, physical properties (such as a molecular weight) and molding conditions (such as pit depth, temperature and molding pressure) of the resin to be used. It is desirable to confirm them in advance by various tests and experiments.

When the thin film of the thermosetting resin is uncured, a precision mold provided with pits having a predetermined arrangement is pressed against it, and is separated, whereby a group of micro pillars each having the intended shape is formed. This is then cured by thermosetting or photo curing, thereby getting a functioning substrate characterized by excellent mechanical strength. When curing the thermosetting resin, it is necessary to study the curing temperature and heating profile in order to select such conditions as to avoid melting, flowing or deforming of the resin. It goes without saying that a curing agent or curing accelerating agent can be added to the thermosetting resin.

The present invention provides a functioning substrate equipped with the micro pillar group of organic polymer that allows free control of dimensions, aspect ratio and others. The present invention uses the organic polymer to manufacture the group of columnar micro pillars; it is possible to use a simple production technique of pressing, which provides a low-priced functioning substrate.

It is easy to form a micro pillar of a small aspect ratio. The present invention allows s simple method to be used to produce a micro pillar in units of nanometer having a big aspect ratio of 4 or more. To put it another way, it provides a functioning substrate in units of nanometer that can be utilized for a great variety of applications. It is also possible to change the distance between center positions of micro pillars (pitch: p) to provide varied functions.

### DESCRIPTION OF THE PREFERRED EMBODIMENTS

### First Embodiment

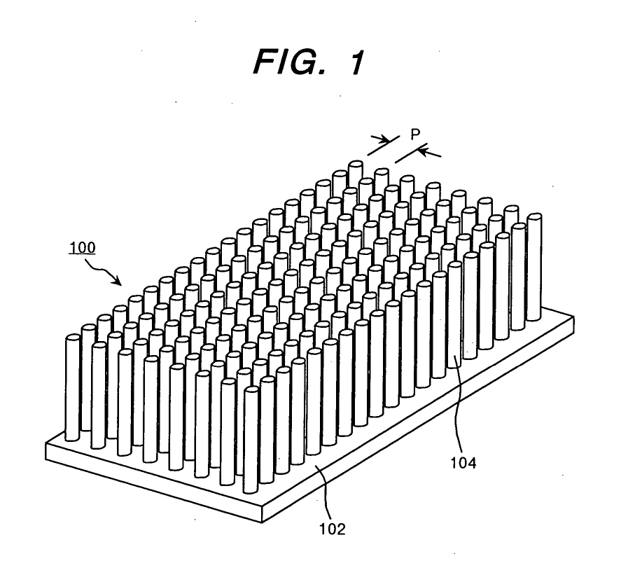

FIG. 1 is a schematic perspective view representing a functioning substrate 100 equipped with a micro pillar group 104 manufactured for the present embodiment. The micro pillar group 104 is made of PMMA, which has a molecular weight of 2,000 through 600,000. The matrix 102 consisting of the same organic polymer as that of the micro pillar group 104 is integral with the micro pillar group, and holds the micro pillar group firmly in position. In FIG. 1, micro pillars of the micro pillar group 104 are arranged in proper alignment both in the longitudinal and transverse directions. They can be arranged on a random basis. Further, the height and aspect ratio of the micro pillar group can be changed in response to the position of the micro pillars. Such changes are one of the characteristics of the manufacturing method of the present invention.

Removing part of the micro pillars can also provide a special function to the functioning substrate. The method according to the present invention allows easy formation of a functioning substrate having a linear defective portion in one process by forming the defective portion in the micro pillars in advance using a precision mold.

### Second Embodiment

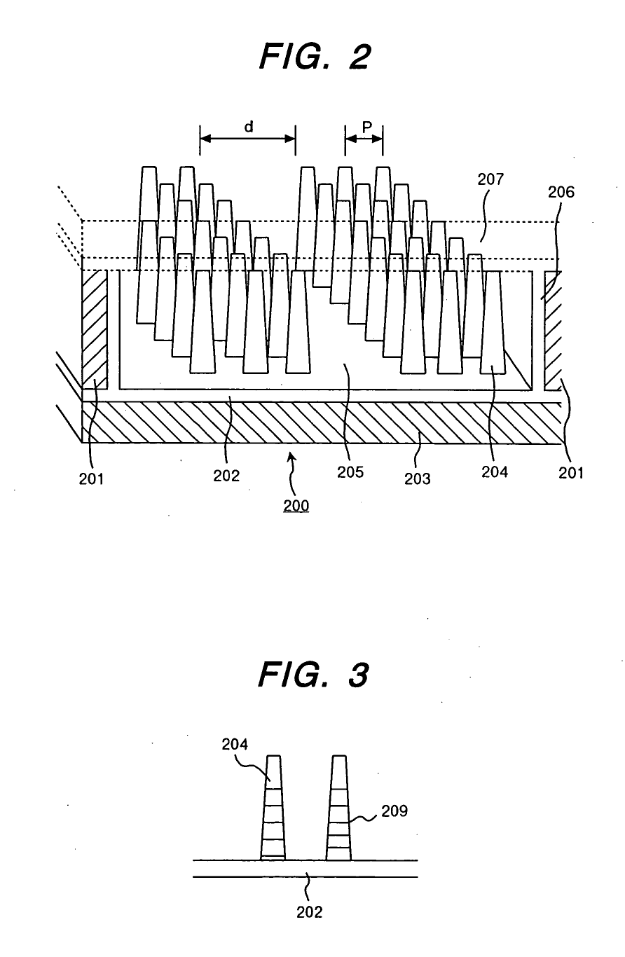

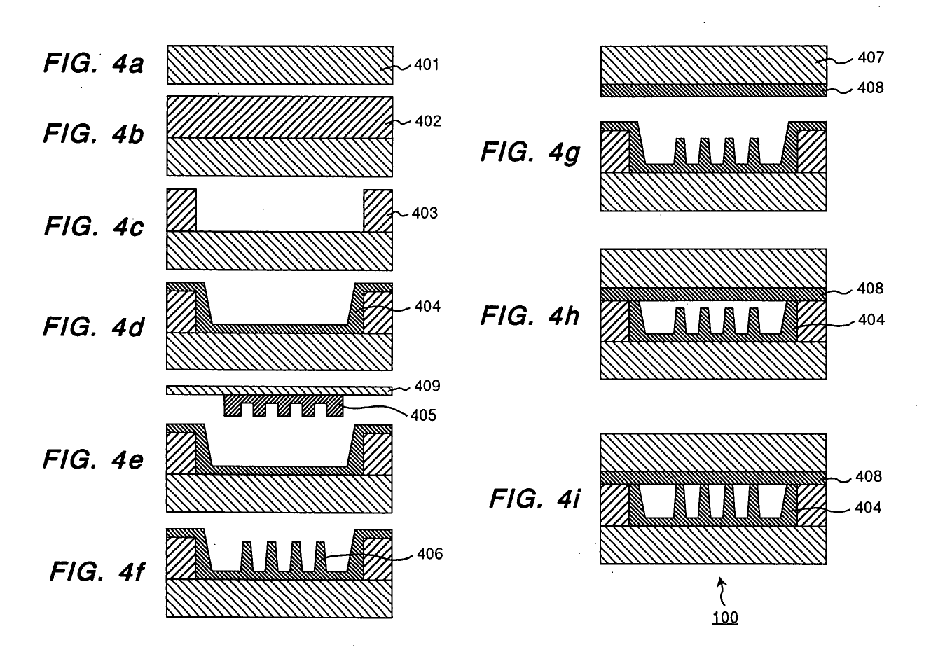

FIG. 2 is a diagram showing the lateral configuration of a micro pillars 204, and FIG. 3 shows a side view of the micro pillars 204. FIG. 4 is a flowchart representing the process of manufacturing a functioning substrate given in FIGS. 2 and 3.

In FIGS. 2 and 3, the height of the columnar micro pillar 204 is 3 μm and the equivalent diameter is 330 nm at the root and 300 nm at an intermediate position. A smooth surface is formed about 1 μm from the top of the columnar micro pillars 204, and a lateral striped pattern 209 is formed on the surface of the portion about 2 μm from the root, as shown in FIG. 3.

The micro pillars 204 are formed at a certain pitch P, and the equivalent diameter on the bottom surface is 300 nm with a height of 3 μm, showing that the aspect ratio on the bottom surface is 9. Further, the equivalent diameter of the micro pillar 204 at the intermediate height is 300 nm, so the aspect ratio at the intermediate position is 10. This is a sufficiently big aspect ratio in excess of 4. The aspect ratio in the sense used in this invention is defined as a value at the intermediate height of a micro pillars unless otherwise specified.

It can be seen that each of the columnar micro pillars 204 has a tip end smaller than the bottom, presenting the flared shape. In this embodiment, the columnar micro pillar tapers from the root toward the tip end. It is also possible to make such arrangements that the micro pillar tapers from the root toward the swelled tip end, for example.

One of the characteristics of the columnar micro pillar of the present invention is that it has a portion tapering from the root toward the tip end. Since the tip end of the micro pillar is smaller than the bottom exhibiting the shape of a folding fan, it is difficult to remove the micro pillars from the supporting member from which the micro pillars are standing. Further, the micro pillar is made of the same material as that of the underlying supporting member so that the micro pillars cannot be easily removed from the supporting member.

As will be described later, the columnar micro pillars 204 are made of PMMA, the same material as that of the underlying film (supporting member) 202. Further, the columnar micro pillars 204 are connected integrally to the underlying film 202. The portion 205 having a specific width “d” is formed by removing a specific row of the micro pillar group. This step provides the specific function. A spacer 201 is formed between the first matrix 203 and second matrix 207, thereby protecting the micro pillar group. A resin film 206 is formed to protect the tip end of the micro pillar group and to ensure the close adhesion with the second matrix 207.

FIG. 2 is a perspective view of an optical device 200 for realizing an optical wave-guide manufactured by the method given in FIG. 4. The optical device 200 comprises;
a spacer 201 of silicon nitride having a height of 3 μm,
a thin film 202 mainly composed of PMMA (with oxidant inhibitor and flame retardant added thereto) having a thickness of 0.5 μm,
a first substrate 203 made of silicon having a thickness of 550 μm and having a width of 1 mm,
a second substrate 207 made of polycarbonate, and
a group of columnar micro pillars 204.

The surface of the spacer 201 is covered with a resin film of the same material as the micro pillars 204 and the film 202.

In the present embodiment, the columnar micro pillar 204 and underlying film 202 are made of PMMA, but can be made of cyclo-olefin polymer, polystyrene, polycarbonate, polyethylene terephthalate (PET), polylactic acid, polypropylene, polyethylene, polyvinylalcohol, ABS resin, AS resin, polyamide, polyacetal, polybutylene terephthalete, glass reinforced polyethylene terephthalate, modified polyphenylene ether, polyvinyl chloride, polyphenylene sulfide, polyetherether ketone, liquid like polymers, fluoride resins, polyacrylate, polystyrene, polyether sulfone, polyamideimide, polyetherimide, thermoplastic polyimide, phenol resins, melamine resins, urea resins, epoxy resins, unsaturatged polyester resins, silicone resinsins, diallylphthalate resin, polyamide bismaleimide resin, polybiamide triazole resin and other resins. Two or more of the materials can be mixed, or photo-activators, oxidation inhibitor agents, flame retardant agents, etc. can be added to the materials.

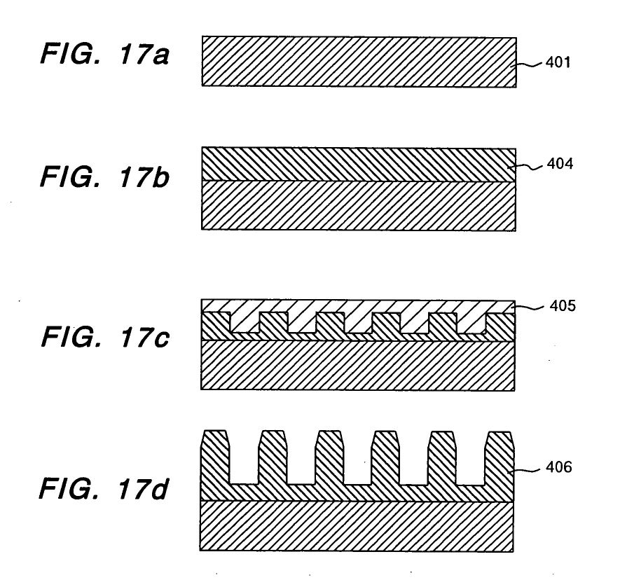

The method of manufacturing the main part of the functioning substrate is shown in FIG. 17. The supporting member 401 is made of silicon wafer having a crystal orientation of 100 and a diameter of 150 mm. The supporting member 401 was covered with a thin film 404 of PMMA having a molecular weight of 120,000, the film being coated by spin coating method. The thickness of the film was 1.5 μm.

Then, a precision mold 405 having pits on the surface thereof was pressed to the film 404. The mold 405 had a diameter of 150 mm and was a silicon wafer having the same crystal orientation as that of the supporting member 401. The mold 405 was lifted vertically to separate it from the film, thereby forming columnar micro pillars 406. As shown in FIG. 17, the aspect ratio of the columnar micro pillars was 4 times that (about 1) of the pits of the mold.

Although it was difficult to form pits of the order of nano meters that have a large aspect ratio, the columnar micro pillars having a large aspect ratio can be easily manufactured by the method of the present invention.

In the present embodiment, the silicon wafer having a crystal orientation (100) and a diameter of 150 mm was used as the supporting member 401. However, the substrate 401 is not restricted to a silicon wafer. For example, an inorganic material such as glass, an organic material such as polycarbonate or their laminated structure can be used as the substrate 401.

In the present embodiment, a PMMA-made thin film 404 was formed on the surface of the supporting member 401. In addition to the PMMA, cyclo-olefin polymer, polystyrene, polycarbonate, polypropylene or such organic polymer can be used for this purpose. Such inorganic material as silica can be added to these polymers.

In the present embodiment, a silicon wafer having a crystal orientation (100) and a diameter of 150 mm was used as the supporting member 401. It is also possible to use the thin film of metal such as nickel or the organic substance such as PDMS.

Further, the diameter and height of the columnar micro pillars 406 can be controlled by adjusting the depth of the concave portion (pit) of the mold 405, or the thickness and viscosity of the thin film 404. The size of the bottom of the columnar micro pillar 406 can be controlled by increasing the opening area of the concave portion of the mold 405. Further, the position for forming the columnar micro pillar 406 can be controlled by adjusting the position of the pit of the mold 405.

Moreover, when a thermoplastic resin is used to make the columnar micro pillar 406, it is also possible to adjust the temperature of the columnar micro pillar 406 and to control the shape of the columnar micro pillar 406 easily. Furthermore, if the photo-cured material to form the columnar micro pillar 406 is used, light is applied at the time of forming the columnar micro pillar 406, thereby ensuring easy control of the columnar micro pillar 406.

The following describes the details of the method for manufacturing an optical device 200. FIG. 4 is a flowchart representing the method for manufacturing an optical device 200. At first, a silicon nitride film 402 having a thickness of 3 μm was deposited on the first substrate 401 made of silicon shown in FIG. 4(a) according to the plasma CVD method, as shown in FIG. 4(b).

Then the silicon nitride film 402 was patterned to form a spacer 403 as shown in FIG. 4(c) according to the method of photolithography. Then the first resin film 404 was formed on the spacer 403 and first substrate is 401 by spin-coating the PMMA (solvent: ethyl cellosolve) obtained by adding 5 percent by weight of ultra fine silica particles having a particle diameter of 2 nm, as shown in FIG. 4(d).

Then the mold (precision mold) 405 shown in FIG. 4(e), where the pit having a depth of 1 μm and a diameter of 500 nm is formed on the surface at a pitch of 1-μm, is pressed against the first resin film 404, and part of the resin film has been pressed into the pit. After that, the group of columnar micro pillars 406 is formed, as shown in FIG. 4(f), by separating the mold 405. At the back face of the mold 405, a plate 409 is provided to make a pressing force to the film even and horizontally, as well as to keep the film and the face of the mold in parallel with each other.

The shape of the group of columnar micro pillars is shown in FIG. 2. The columnar micro pillars 204 are arranged to have a height of 3 μm and a cycle (pitch p) of 1 μm. The equivalent diameter is about 300 nm at the intermediate position in the direction of height of the columnar micro pillar, and 330 nm at the root. As shown in FIG. 3, the portion about 1 μm on the upper part of the columnar micro pillar 204 is formed to have a smooth surface, and the surface of the portion about 2 μm from the root has a striped pattern in some cases.

Further, the equivalent diameter of the intermediate height of the columnar micro pillar 204 is 300 nm, and the height is 3 μm, so it is apparent that the ratio of height to one side (aspect ratio) is 10, and is sufficiently greater than 4. It is also apparent that the sectional area of the tip end of the columnar micro pillar 204 is small, with the shape of a folding fan exhibited. Further, the columnar micro pillar 204 is made of PMMA, the same material as that of thin film 202. The columnar micro pillar 204 is bonded integrally to the thin film 202.

Then the second resin film 408 having the same component as that of the resin film 404 was formed on the second substrate 407 of polycarbonate, as shown in FIG. 4(g) according to the spin coating method. This was followed by the step of laying the second substrate 407 thereon, as shown in FIG. 4(h). After that, a pressure of 10 MPa was applied to the first substrate 401 and second substrate 407, and heating was provided at 150 degrees Celsius for two minutes, thereby getting an optical device 200 shown in FIG. 4(i). It should be noted that, in the process of FIG. 4(h), the resin film 404 was integrally bonded with the resin film 408.

The columnar micro pillar 204 was integrally bonded with the thin film 202. The micro pillar 204 is arranged at a cycle (pitch p) of 1 μm so that a photonic band gap appears. Further, the columnar micro pillar 204 is not arranged in the path for allowing light to pass through. A gap (optical path) 205 having a width of “d” is formed.

The columnar micro pillar of the present invention need not always be formed in the shape shown in FIG. 2. When the group of columnar micro pillars is formed according to the press mold method for thermoplastic resin film to be described below, the approach shown in FIGS. 2 and 3 is commonly practiced. To put it another way, the top end of the micro pillar 204 has a smaller sectional area than the bottom end. As shown in FIG. 3, the top of the micro pillar is smooth, but the bottom is provided with several lines 209 in the lateral direction in some cases. The resin for generating these lines is not clear, but this is one of the configuration characteristics of the columnar micro pillar according to the press mold method.

For simplicity, only seven micro pillars 204 are described in one row in FIG. 2. In the optical device 200 manufactured on the tentative basis, 80 micro pillars 204 are formed in one row.

Light was applied from the end of the optical device 200 having a length of 30 centimeters, and the spectrum of transmitted light was checked. The result of this test has revealed that light having a wavelength of 1.8 μm is transmitted. Transmittance was 80 percent.

In this present embodiment, the main components of the micro pillar 204 and thin films 404 and 408 were PMMA. It is also possible to use other thermoplastic resins, for example, the high molecular material including cyclo-olefin polymer, polystyrene, polycarbonate, polyethylene terephthalate (PET), polylactic acid or polypropylene. Further, refractive index can be changed by adding ultra fine particles such as silica and gold to the high molecular material. It is also possible to bond other monomer on the surface of the formed columnar micro pillar or to provide chemical modification by plating. The waveform of transmitted light can be adjusted by changing the equivalent diameter and cycle. That this adjustment is possible is the feature basically different from the art described in the aforementioned non-patent document 1. Application to communications device conforming to various communications wavelength is enabled by adjusting the equivalent diameter and cycle of the columnar micro pillar 204 in conformity to the communications wavelength. Further, photo-cured resin can be used. In this case, the columnar micro pillar is hardened during press formation and after molding.

As described above, the columnar micro pillar 204 of the optical device 200 is formed by press molding. This method does not require dry etching or photolithography, unlike cases of a prior art optical device or semiconductor process. This method has the advantage of producing an optical device 200 at lower costs.

Further, in the prior art columnar micro pillar made of silicon root material, the micro pillar was observed to have been damaged when pressure was applied to the second substrate 407 and first substrate 401 placed in combination as shown in FIG. 4(h). In the present embodiment, however, little damage is observed even when the columnar micro pillar 406 is brought in contact with the resin film 408, since high molecular material is used as the major component of the micro pillar 204. To put it another way, close adhesion with the resin film 408 and fixing in position are ensured without the columnar micro pillar 204 being damaged. Further, incoming light does not leak from the gap between the columnar micro pillar 204 and second resin film 408. This improvement of adhesion has reduced the background noise of the output light.

The optical device requires the micro pillar to be formed on a periodic basis in order to ensure that the photonic band gap appears. The photonic band gap can be obtained by forming the areas with different refractive indexes at a cycle half the light wavelength. Accordingly, the wavelength of the transmitted light can be controlled by adjusting the space (pitch) between columnar micro pillars. To transmit the light of short wavelength on a selective basis, it is necessary to reduce the space between columnar micro pillars and to reduce the equivalent diameter or one side of the columnar micro pillar.

The height of the columnar micro pillar is related to the aperture for admitting incoming light to be used in the optical device, so this height is preferred to be at least several μm. The method of manufacturing the micro pillar in the present embodiment provides the advantage of ensuring a sufficient intensity of incoming light because it allows the aspect ratio of the columnar micro pillar to be increased.

According to the present embodiment, the tip end of the columnar micro pillar is smaller than the bottom, showing the shape of a folding fan. This feature makes it difficult to remove the group of columnar micro pillars from the substrate. Moreover, the group of columnar micro pillars is made of the same material as the underlying material. This feature also makes it difficult to remove the group of columnar micro pillars from the underlying material. The optical device can be easily handled because the micro pillar is integral with the substrate.

### Third Embodiment

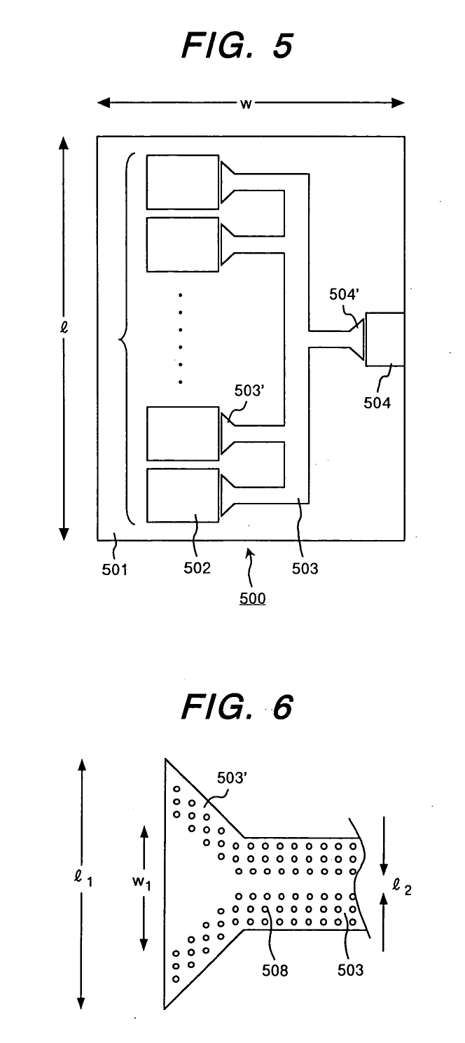

The present embodiment refers to an example of applying the optical device 200 for changing the traveling direction of incoming light, to the optical information processing apparatus. FIG. 5 is a schematic block diagram of the optical circuit 500 manufactured by the present invention. The optical circuit 500 is composed of ten transmission units 502 consisting of indium-based semiconductor lasers and driver circuits, optical wave-guides 503 and 503′, and optical connectors 504 and 504′ arranged on the substrate 501 of aluminum nitride having a length (I) of 30 mm, a width (w) of 5 mm and a thickness of 1 mm. The transmission wavelengths of the ten semiconductor lasers are each made different in the range from 2 through 50 nm. The optical circuit 500 is a basic component of the optical multiplex communications system device. The present invention is applied to the optical wave-guides 503 and 503′. In the optical wave-guide 503, the optical signal inputted from the transmission unit 502 is received by the oscillation section 503′ of the connecting section and is sent to the optical connector 504 from the connection section 504′ through the sub-wave-guide 506 and main wave-guide 505 sequentially. In this case, the optical input signal is formed by merging of sub-wave-guides.

The configuration of the oscillation section 503′ is shown in FIG. 6. The connection section 504′ is designed in a configuration as a mirror image of the one given in FIG. 6.

FIG. 6 is a schematic layout diagram representing a columnar micro pillar 508 inside the optical wave-guide 503. To permit misalignment between the transmission unit 502 and optical wave-guide 503, the width of the input section of the optical wave-guide 503′ is 20 μm, and the plane section is configured in a flared form. In the straight central portion, one row of the group of columnar micro pillars is removed to form an area free of any photonic band gap. This allows the signal light to be led to the area (w₁) having a width of 1 μm. It should be noted that the space (pitch) between columnar micro pillars 508 is 0.5 μm. For simplicity, the number of the columnar micro pillars 508 is smaller than the actual number in FIG. 6.

The optical circuit 500 makes it possible to output the optical signals having ten different types of wavelength in a superimposed form. Since the traveling direction of the light can be changed, the lateral width of the optical circuit 500 can be as small as 5 mm, thereby reducing the size of the device for optical communications. Since a columnar micro pillar 508 can be formed by the mold pressing method, the production cost is reduced. The device of the present embodiment superimposes inputted beams of light. It is apparent that the optical wave-guide 503 is useful to all optical devices that control an optical path.

As described in the aforementioned embodiment, the present invention provides an optical device on the order of nanometers that allows free selection of a desired wavelength of the light used as an optical signal, by application of the film equipped with columnar a group of columnar micro pillars according to the present invention. The present embodiment provides easy formation of a photonic band gap and micrometer-sized or nanometer-sized optical path when the organic polymer or material mainly composed of organic polymer is used to form the columnar micro pillar group on the order of nanometers.

When the aforementioned mold is pressed against the thin film of the thermoplastic resin, and the film is separated, the resin pressed into the pit is stretched, thereby forming a group of micro pillars whose equivalent diameter at the root is slightly smaller than the pit diameter but greater than the pit depth. The equivalent diameter and length of the micro pillar depend on the type, physical properties (molecular weight) and molding conditions (pit depth, temperature, molding pressure, etc.) of the resin used. Accordingly, it is preferred that various experiments be conducted in advance for verification. In any case, this condition setting determines the conditions for getting the micro pillar group, and the optimum photonic crystal i.e. optical device can be obtained from the relationship with the wavelength of the signal to be used.

The optical device of the present invention is characterized as follows: the columnar micro pillars are arranged according to a predetermined layout method where the organic polymer is used as the main component. An optical path is constituted according to the photonic band gap to input or output the optical signal. Further, the micro pillars consisting of the organic polymer are arranged in the space sandwiched between two opposing substrates and this substrate according to a predetermined layout method, thereby constituting the photonic band gap. It is preferred that the micro pillar be connected to the thin film formed on the surface of the substrate. It is especially preferred that the material of the thin film formed on the substrate surface be the same as that of the micro pillar. A predetermined refractive index can be provided by adding at least of one type of the material selected from the fine particle group of oxide nitride and metal, to the aforementioned organic polymer. It is also preferred that two opposing substrates and space separating between these substrates be provided to support the micro pillar group.

Here the equivalent diameter (diameter or one side) of the micro pillar can be adjusted freely in the range from 10 nm through 10 μm, root on the relationship with the wavelength of the light source used for the semiconductor laser or the like. The height of the micro pillar is preferred to be 50 nm through 10 μm. The distance (pitch) between the micro pillars is about half the wavelength of the signal to be used.

### Fourth Embodiment

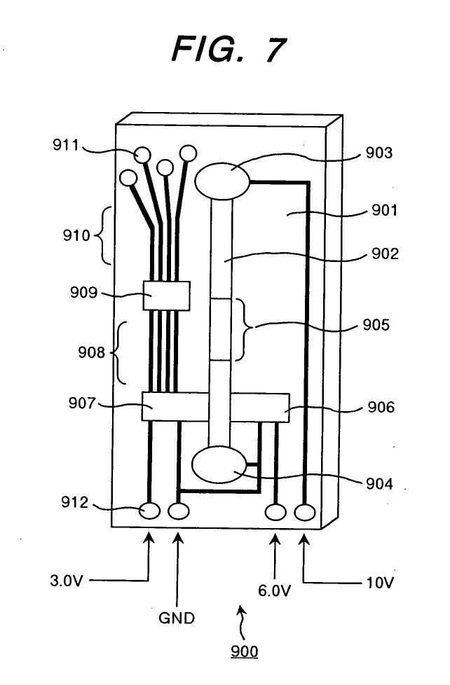

In the present embodiment, the micro pillar assembly 100 shown in the first embodiment is applied to the biochip 900. FIG. 7 is a schematic plan representing the biochip 900. A flow path having a depth of 3 μm and a width of 20 μm is formed in the glass-made substrate 901. The sample containing DNA (deoxyribonucleic acid), blood, protein and others is led into the inlet 903, and is sent towards the outlet 904 through the flow path 902.

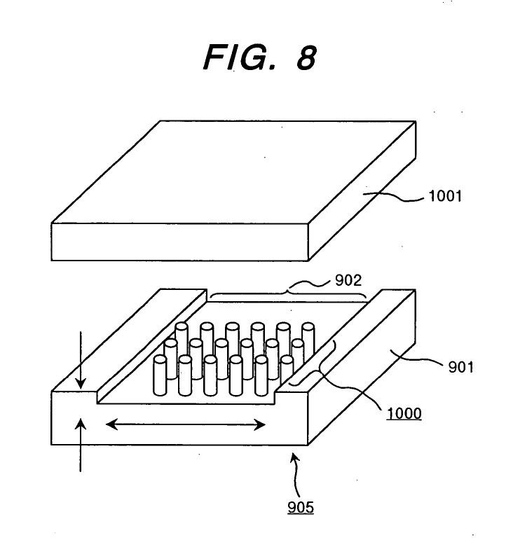

A molecular filter 905 is installed in the flow path 902. A micro pillar group 1000 (FIG. 8) having a diameter of 250 through 300 nm and a height of 3 μm is arranged in the molecular filter 905. FIG. 8 is a perspective view of the molecular filter 905. A flow path 902 is formed on the substrate 901. The micro pillar group 1000 is formed in part of the flow path 902.

The substrate 901 is covered by the upper substrate 1001, and the sample moves inside the flow path 902. In the analysis of a DNA chain length, the DNA-containing sample is separated according to the DNA chain length when passing through the molecular filter 905 in the flow path 902 by electrophoresis. To put it another way, the DNA of shorter chain passes through the molecular filter faster than the DNA of longer chain. This allows the DNA to be separated according to the chain length.

From the semiconductor laser 906 mounted on the surface of the substrate 901, laser light is applied to the sampled having passed through the molecular filter 905. When the DNA passed by, the incoming light to the optical detector 907 is reduced about 4 percent. This allows the DNA chain length to be analyzed using the output signal from the optical detector 907.

The signal detected by the optical detector 907 is inputted into the signal processing chip 909 through the signal wiring 908. The signal processing chip 909 is connected with the signal wiring 910, which is connected with the output pad 911. This is connected to an external terminal. The power is supplied to each component from the power supply pad 912 installed on the surface of the substrate 901.

The molecular filter 905 in the present embodiment is composed of the substrate 901 equipped with a concave portion, many micro pillars formed on the concave portion of the substrate 901 and an upper substrate 1001 formed to cover the concave portion of the substrate 901. Here the tip end of the micro pillar is formed so as to contact the upper substrate.

An organic polymer is the major component of the micro pillar group 1000, which can be deformed. This protects the upper substrate 1001 against damage when the flow path 902 is covered with the upper substrate 1001, and allows the upper substrate 1001 and to come in close contact with the micro pillar group 1000. This configuration prevents the sample from leaking through the gap between the micro pillar group 1000 and upper substrate 1001, thereby ensuring highly sensitive analysis.

When the DNA was subjected to chain length analysis, the resolution of the root pair was ten root pairs in terms of half-width in the case of a glass-made micro pillar group; whereas the resolution of the root pair was three root pairs in terms of half-width in the case of a micro pillar assembly 1000 of organic polymer. This test has revealed that such an improvement can be achieved. In the molecular filter of the present embodiment, the micro pillar group is brought into direct contact with the upper substrate. It is also possible to make such arrangements that the film of the same material as that of the micro pillar group is formed on the upper substrate, and the micro pillar group and this film are kept in contact with each other, for example. This arrangement improves adhesion.

In the present embodiment, only one flow path 902 is used. Simultaneous multiple analyses can be made by forming a plurality of flow paths 902 by providing micro pillars of different sizes. In the present embodiment, a DNA is used as a sample for analysis. If a molecule reacting with sugar chain, protein or antigen is modified on the surface of the micro pillar group 1000 in advance, it is possible to analyze the sugar chain, protein or antigen. In this manner, sensitivity of immunity analysis can be improved by modifying antigen on the surface of the micro pillar.

The present embodiment allows simple formation of a micro pillar group of organic polymer for analysis on the order of nanometers. It also permits the position diameter, height and aspect ratio of the micro pillars of organic material to be controlled by adjusting the irregularities on the mold surface and the viscosity of the thin film of organic material. It further provides a micro-biochip of high sensitivity for analysis.

In the micro-biochip in the present embodiment, it is preferred that multiple micro pillars be arranged on the sample detection section, these micro pillars be made of organic polymer and the diameter of these micro pillars be 10 nm to 100 μm with a height of 0.5 through 500 μm. The diameter or one side of the micro pillar is preferred to be 10 nm through 100 μm, the same size as that of the biopolymer or cell. The height of the micro pillar is adjusted to the height of the micro-chip flow path. A microchip of high sensitivity for analysis can be created by interaction with the biopolymer (especially biopolymer) and cell if the surface area for reaction is increased by providing multiple micro pillars on the microchip detecting section.

In the micro pillar in the present embodiment, the ratio of the height to the diameter or one side is sufficiently greater than 1; therefore, micro pillars can be arranged in the microscopic flow path having a depth of several microns or more.

### Fifth Embodiment

### Cell Culture Sheet

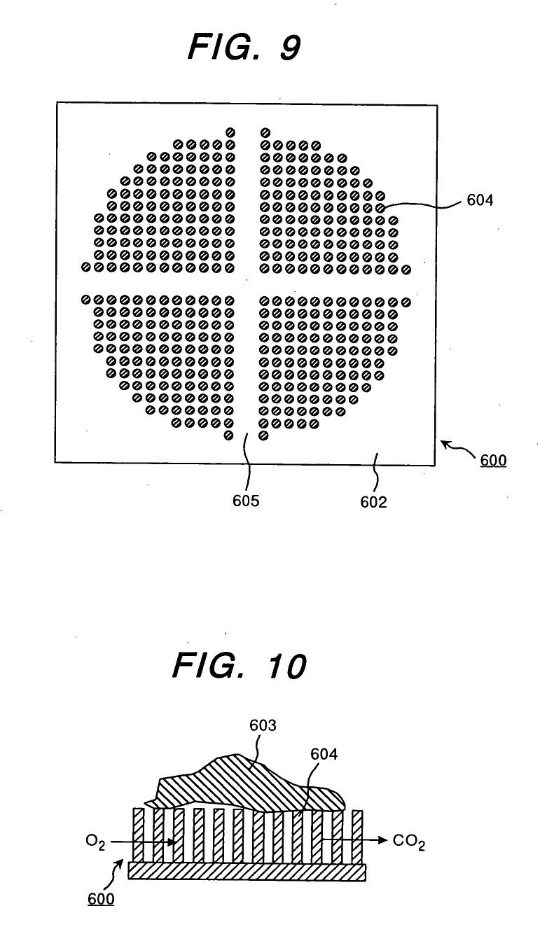

FIG. 9 is a plan representing a cell culture sheet 600. The cell culture sheet 600 is a thin film 602 mainly composed of a 0.5 μm-thick PMMA, and a group of columnar micro pillars 604 having an equivalent diameter of 2 μm mainly composed of a 0.5 μm-thick PMMA, where the group of columnar micro pillars 604 extends from the thin film.

A gap 605 is formed by removing part of the group of columnar micro pillars. This cell culture sheet is placed in a glass-made Petri dish and other vessel, in which culture solution is placed. As shown in FIG. 10, a cell or the like is cultured by placing such a cell (tissue) as a skin, bone or blood, culturing media and culture solution 603 including nutrient on the group of columnar micro pillars 604 of the cell culture sheet 600.

It is preferred that the group of columnar micro pillars be provided with a gap 605 of a certain size formed by removing part of the columnar micro pillar. In the present embodiment, gaps are arranged in a cross form, as shown in FIG. 9. Formation of such gaps ensures easy flow of the culture solution and an effective supply of nutrient the cell. It also permits efficient removal of a waste material from the cell at the time of cell culture.

It has been proven that use of this cell culture sheet substantially reduces the damage of the sheet-like epidermal cell caused by separation from the Petri dish when a conventional glass Petri dish is used. Further, it has been possible to improve the plating efficiency in the transplantation of sheet-like epidermal cells onto the rabbit skin. The culture solution can easily flows to the entire sheet-like epidermal cell through the gap formed on the bottom of the epidermal cell created by the columnar micro pillar on the cell culture sheet, thereby ensuring efficient supply of nutrient to the cell or efficient removal of waste material from the cell. This has resulted in reducing the death of the epidermal cell during cell culture that has often occurred so far.

The following describes how to produce the cell culture sheet: The PMMA (solvent: ethyl cellosolve) is applied on the mold side of the stage of a press device, thereby forming a resin film. A mold with a great number of pits each having a depth of 1 μm and a diameter of 500 nm at a pitch of 1 μm formed on the surface, is pressed against the resin film, and part of the resin film is pressed into the pit. Then the mold was separated to form a group of columnar micro pillars so as to get a cell culture sheet with a group of columnar micro pillars formed on the thin film. In the present embodiment, a resin film is applied directly on the stage. It is also possible to form the cell culture sheet by applying the resin film on the substrate of silicon or the like.

The group of columnar micro pillars has a height of 3 μm, and micro pillars are arranged at a cycle of 1 μm (pitch). The equivalent diameter of the columnar micro pillar is 300 nm at the intermediate position in the direction of height and is 330 nm at the root. Accordingly, the aspect ratio of the micro pillar is 10. The portion about 1 μm on the upper part of the columnar micro pillar is formed to have a smooth surface, and the surface of the portion about 2 μm from the root has a striped pattern in some cases. Further, the sectional area of the tip end of the columnar micro pillar is smaller than the bottom, showing the shape of a folding fan.

The cell culture sheet formed in the present embodiment was placed in the glass-made Petri dish with culture solution therein, and the normal human epidermal cornified cell (used medium: HuMedia-KB2 (by Kurabo Inc.) at 37° C. under the flow of 5% CO₂) was cultured on the cell culture sheet according the conventional method. In this experiment, epidermal cornified cells were correctly attached to the cell culture sheet and proliferated in the form of a sheet.

Fourteenth day after starting the culture, the cultured cell was covered with a polyvinylidene difluoride (PVDF) film having a diameter of 2 cm, and the medium was drawn by suction, whereby the sheet-like epidermal cornified cell grown on the nano-pillar sheet was separated from the cell culture sheet together with the PVDF film. This sheet-like epidermal cornified cell could be removed from the covered PVDF film easily. Damage to the sheet-like epidermal cornified cell caused by separation from the cell culture sheet could be substantially reduced as compared to the case where the conventional glass-made Petri dish was used.

It is also possible to provide the high molecular material with hydrophilic treatment by plasma treatment. The high molecular material is not restricted to a particular type, but it is preferred that the selected material do not give a serious effect to the cell (tissue) to be cultured. For example, selection of polystyrene, PMMA, or polylactic acid is preferred.

In the present embodiment, the tip end of the columnar micro pillar has a tip end smaller than the bottom, presenting the shape of a folding fan. This configuration prevents the group of columnar micro pillars from being removed from the substrate. Since the group of columnar micro pillars is made of the same material as the underlying material, the group of columnar micro pillars is not easily removed from the substrate. Further, handling of the cell culture sheet is facilitated by the integral configuration of the micro pillars with the substrate.

### Sixth Embodiment

### Water Repellent Sheet

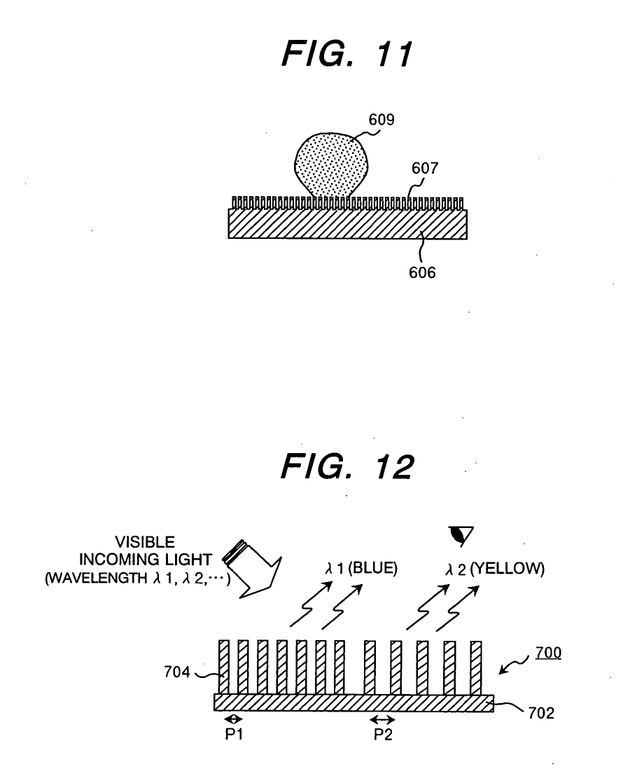

The following describes a further embodiment of the present invention: The present embodiment shows the case of using a thin film (sheet) as a water-repellent film, wherein the thin film is used as a group of columnar micro pillars is formed on the surface. As shown in FIG. 11, the water-repellent film 605 of the present embodiment consists of a group of columnar micro pillars 607 formed on the sheet 606. In the columnar micro pillar of the present embodiment, it is preferred that the pitch (gap) between micro pillars be from 20 nm through 10 μm with the height ranging from several μm through several tens of μm, and the diameter of the tip end of the micro pillar be from 50 through 500 nm. The material of the columnar micro pillar is not restricted to a particular type, but it is preferred to use a water-repellent material such as PMMA. Alternatively, it is also possible to treat the surface of the micro pillar group with water-repellent agent.

In the water-repellent film of the present embodiment, the contact angle of a water drop 609 can be increased by forming a group of columnar micro pillars 607 on the surface, as compared to the case of using a water-repellent sheet 606 as a single body, as shown in FIG. 11, and the water repellent property of the material can be made amplified by “lotus leaf effect” obtained from formation of the group of columnar micro pillars 607. Since the water-repellent group of columnar micro pillars of the present embodiment can be arranged in a closely packed configuration on the order of nanometers, it is not easily crushed or removed. It is also characterized by persistent water- and oil-repellency.

The water- and oil-repellent oil film of the present embodiment can be applied to the surface of an umbrella, clothing and wall, for example. In this case, a water-repellent film is produced as follows: A sheet is manufactured according to the same method as that used in the fifth embodiment, wherein this sheet has a micro pillar group formed thereon. Then this sheet is laminated using a pressure sensitive adhesive. Another method is to transfer the sheet directly on the surface of the matrix of the umbrella, clothing or wall where water- and oil-repellent oil film is to be formed.

The water-repellent film of the present embodiment eliminates the need of water-repellent treatment that has been commonly used in the prior art method. It provides simple modification of the material surface in one transfer operation.

### Seventh Embodiment

### Color Sheet Free of Coloring Agent

The following describes the seventh embodiment where the group of columnar micro pillars formed on a sheet is used as a color sheet free of coloring agent or a non-dyed color producing sheet. FIG. 12 shows the color producing principle of the color sheet free of coloring agent. The visible light entering the columnar micro pillar is reflected while causing interference in the group of columnar micro pillars. This appears as if the sheet surface were of coloring. As shown in FIG. 12, the interference of the visible light can be varied by changing the spaces (pitches) P1 and P2 between the micro pillars, with the result that the visible color tone is changed.

For example, when the white light containing the components of wavelength λ1, λ2 . . . is applied to the micro pillar group 704 of the sheet 700, the light reflected from smaller pitch “P1” is turned into the light of λ1 (blue), while the light reflected from greater pitch “P2” is turned into the light of λ2 (yellow).

As the space between micro pillars is increased, the interference of the color having a greater wavelength is intensified in such a way that the color is changed in the order of blue, green, yellow and red. Adjusting the space between micro pillars in this manner allows the sheet to appear as if it were colored, without using a dye or pigment. It is also possible to change the color according to each place. Further, since the coloring sheet free of coloring agent uses coherent light, the color tone is changed according to the angle of viewing.

In the similar manner, the interference of visible light can be changed by adjusting the diameter and height of the micro pillars. The color tone of the sheet can be changed by adjusting the diameter and height of the micro pillars.

In the non-dyed coloring sheet of the present embodiment, to ensure that the interference of visible light will occur, the diameter of the columnar micro pillar to be used is preferred to be 100 through 2000 nm, and the space between micro pillars is preferred to be 100 through 2000 nm, with the height of the micro pillar ranging from 1 μm through 5 μm.

The non-dyed coloring sheet of the present invention finds application in clothing, on-board sheet, accessories, wall paper, holography, etc.

### Eighth Embodiment

### Anti-Reflective Layer

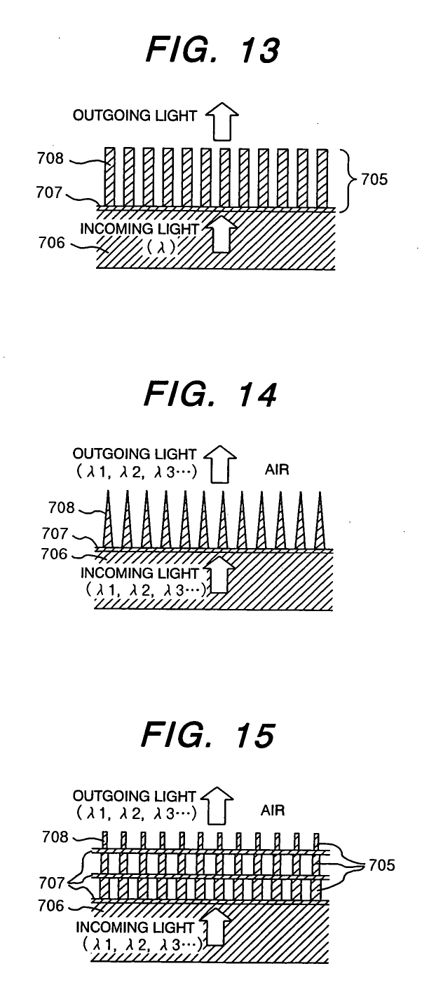

The following describes the eighth embodiment where the group of columnar micro pillars is used as an anti-reflective layer. As shown in FIG. 13, the anti-reflective layer 705 comprises a thin film (sheet) 707 mainly composed of the PMMA having a thickness of 0.1 μm or less, and a group of columnar micro pillars 708 with an equivalent diameter of 1 μm or less, mainly composed of the PMMA, extending from the thin film 707. This anti-reflective film is formed on the surfaces of the display cover glass and optical substrate for optical communications, and is used as an anti-reflective layer. The anti-reflective layer for the display cover glass and optical substrate for optical communications, and is manufactured in two ways; by using a pressure sensitive adhesive to laminate the sheet with a group of columnar micro pillars formed on a thin film, and by applying resin directly on the substrate such as the cover glass and optical substrate and forming a columnar micro pillar according to transfer method, thereby creating a anti-reflective layer. When the sheet is laminated by the pressure sensitive adhesive is used for sheet, the pressure sensitive adhesive to be used is preferred to be the material characterized by excellent optical transparency such as acryl based adhesive.

For the anti-reflective layer of the present embodiment, the effective refractive index in the layer composed of the micro pillar group (hereinafter referred to as “reflective index”) can be adjusted, as desired, by adjusting the diameter, space, height and sectional form of the micro pillar, and changing the ratio between the dielectric area of the micro pillar in the layer composed of the micro pillar group and the air area free of any micro pillar. This allows suppression of the light reflected on the surface of the substrate.

The ideal conditions for the refractive index (n_{AR}) and thickness (t_{AR}) for this the anti-reflective layer can be represented by the following formula:
Reflective index: n_{AR}=(n_{sub}·nₐᵢᵣ)^{1/2}
Thickness: t_{AR}=λ/(4n_{AR})
where the “n_{sub}” denotes the refractive index of substrate, “nₐᵢᵣ” the refractive index of air, and “λ” the designed wavelength.

For example, when the refractive index of the substrate (glass is assumed) is 1.5 and λ is 1.5 μm, the refractive index of the anti-reflective layer is 1.22 and the thickness is about 320 nm, according to the above formula. In this way, the parameter required for the anti-reflective layer is calculated and the shape and arrangement of the micro pillars are adjusted, thereby creating the anti-reflective layer of the display cover glass and optical substrate for communications.

The following describes the structure and functions given in FIG. 13. The light emitting devices are arranged on the side of the substrate 706. The anti-reflective layer 705 is provided to ensure that the light coming from the substrate 706 reduces the reflected light produced on the boundary between the substrate 706 and air. The operating principle of the anti-reflective layer 705 will be described below: In the first place, there is a difference in the refractive index on the boundary between the substrate 706 and the anti-reflective layer 705, so the reflected light #1 to go back to the substrate 706 is produced. Further, reflected light is emitted also on the anti-reflective layer 705 and air, and the reflected light repeats multiple reflections in the anti-reflective layer 705 and transmission from the anti-reflective layer 705. After that, reflected light #2 to return to the substrate 706 is formed. In this case, when the amplitudes of the reflected light #1 and #2 are equal to each other and their phases are opposite to each other, two types of reflected light cancels each other, with the result that the reflected light to return to the substrate 705 is lost.

For transmitted light on the air side, a transmittance of 100% can be obtained when transmitted light #1 having passed directly through the anti-reflective layer 705 is in the same phase with the transmitted light #2 formed after repeating multiple reflection in the anti-reflective layer 705 and transmission from the anti-reflective layer 705.

The conditions of the anti-reflective layer for meeting the aforementioned reflection conditions (full transmission conditions) can be represented by the aforementioned formula. Even when light has launched from the side of air, it is possible to get the same anti-reflective effect as that when light is applied from the substrate 706. The configuration given in FIG. 13 indicates an anti-reflective layer using the phase interference of the light, so the anti-reflective effect will be reduced if the incoming light is deviated from the design wavelength.

The following describes the structures given in FIGS. 14 and 15. They are different from the structure of FIG. 13 in the following points: It is possible to remove the discontinuous surface resulting from the difference in refractive index and to reduce reflection, by slowly changing the refractive index in the anti-reflective layer 705 from the refractive index of the substrate 705 to that of air. Since the phase interference of light is not used, the dependence of refractive index on wavelength can be reduced.

According to the present embodiment, the area of air in the layer formed by the micro pillar group is expanded to realize the layer having a refractive index of as low as 1.3 or less. For example, it is possible to constitute a single-level anti-reflective layer for reducing the light reflected from the surface of the glass substrate having a refractive index of about 1.5. To put it more specifically, when the prior art MgF₂ continuous film is used, the refractive index is about 1.3 when it is small. By contrast, in the anti-reflective layer of the present embodiment, refractive index can be adjusted to about 1.2—a value close to the ideal value—by optimizing the diameter and space of the micro pillars.

FIG. 14 will be used to describe another type of the anti-reflective layer of the present embodiment. In this example, the micro pillar of the group of columnar micro pillars 708 is designed as tapering from the root toward the tip end. As a result of this structure, in the area with a wavelength from 300 nm through 700 nm, the refractive index is reduced to 1/50 or less of that when there is no anti-reflective layer. As described above, the tapered micro pillar allows the refractive index in the layer to be changed in the direction of depth, thereby ensuring an excellent anti-reflective effect in the wide wavelength band.

FIG. 15 is used to describe a further type of the anti-reflective layer. In this example, the sheets 705 with a group of columnar micro pillars 708 having different refractive indexes are laminated on the supporting member. Here the sheets 705 can be laminated as follows: Single-layer sheets can be laminated using a pressure sensitive adhesive. Alternatively, single-layer sheets can be laminated using compression (e.g. a load of 300 kg/cm² heated at 90 degrees Celsius).

Similarly to the tapered configuration given in FIG. 14, the refractive index in the direction of depth can be changed by laminating the sheets having different effective refractive indexes as in the present embodiment. This provides anti-reflective effect in the wide wavelength band.

In addition to the embodiments described above, single-level anti-reflective layers having different wavelength characteristics on the same substrate can be arranged in a desired area by changing the diameter and space of the micro pillars for each specific area on the substrate.

### Ninth Embodiment

### High-Sensitive Electrode

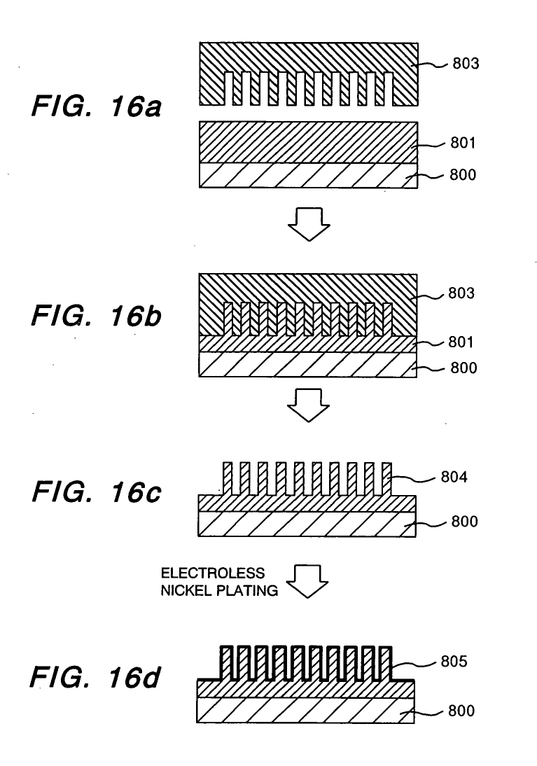

The following uses FIG. 16 to describe the ninth embodiment where a nickel thin film layer is formed on the surfaces of the columnar micro pillars according to the electroless plating method, and this is used as a high-sensitive electrode in the anode stripping method, which is a technique for analyzing a trace quantity of metal ions.

In the first place, polystyrene resin layer 801 was formed on a glass substrate having a length of 30 mm, a width of 30 mm and a thickness of 1 mm, using the same method as disclosed in the first embodiment. Then a precision mold 803 was used to produce a substrate having columnar micro pillars 804 of polystyrene resin on the surface of the polystyrene resin layer 801. The columnar micro pillars 804 formed in this step had an equivalent diameter of 240 nm, a top diameter of 200 nm and a height of 1 μm, where the center-to-center pitch of the columnar micro pillars 804 is 500 nm.

Then the surface of a substrate having micro pillars of polystyrene on the surface was coated with a 20 nm thick nickel layer by the electroless plating. The following describes the electroless plating method used in the present embodiment. In the first place, palladium as a nucleus for electroless plating deposition was attached onto the surface of the substrate having columnar micro pillars of polystyrene, using the processing solution, Neogant of Atotech Japan Inc. This process is generally called “catalytic activation process”. The obtained substrate was immersed in the electroless nickel plating solution, Top Chemialloy 66 of Okuno Seiyaku Inc. for 3 minutes so that a nickel layer was formed on the surface of the substrate having columnar micro pillars on the surface. The obtained substrate surface had a metallic luster. It was observed by a scanning electron microscope to verify that the top diameter of the micro pillar was 240 nm, and the bottom diameter was 280 nm, with a 20 nm-thick nickel layer uniformly formed on the surface of the columnar micro pillar.

The following describes the method of using the anode stripping process to analyze a trace quantity of copper ion contained in an aqueous solution wherein the substrate having on the surface the columnar micro pillars coated with a nickel layer created according to the aforementioned method is used as an electrode. The anode stripping method provides a technique of reducing and concentrating on the electrode the metal ion contained in the solution, and applying anode polarization to the electrode to cause reduction and concentration, thereby identifying the ion in solution based on the potential and flowing electric energy at that time. In the first place, 4 types of copper sulfates were prepared, where copper ion concentrations were 1×10⁻⁸ mol/l, 1×10⁻⁹ mol/l, 1×10⁻¹⁰ mol/l and 1×10⁻¹¹ mol/l, using ultra pure water.

This step was followed by measurement of aqueous solution of copper sulfate conducted according to the anode stripping method, using;
a substrate having on the surface the columnar micro pillars coated with nickel layer, and
a substrate as a comparative example, without columnar micro pillar on the surface thereof coated with nickel layer, prepared according to the aforementioned method and technique, except for the process for forming a columnar micro pillar. In the measurement, a glass beaker was used and the electrode size was 5 mm square. Further, a platinum electrode was used as the counter electrode, and a saturated electrode as a reference electrode. 30 cc of aqueous solution of copper sulfate was poured in the beaker. After the electrode, counter electrode and reference electrode were immersed, purging was carried out by argon gas.

This was followed by the step of applying the potential of −0.4 Vvs. SCE for fifteen minutes to the electrode so that copper was deposited on the electrode surface. Then the potential of electrode was swept at 0.1 volt/min. from −0.4 Vvs. SCE to +1.0 Vvs. SCE, thereby melting the copper deposited on the surface of the electrode. The potential and current at that time were recorded. The obtained result is shown in Table 1. The ratio of electric charge for dissolving the copper of the plate electrode (electrode without micro pillars), to electric charge for dissolving the copper of the highly sensitive electrode substrate (electrode having micro pillars) of the present invention was 10 through 15 independently of copper ion concentration. It has been revealed that measurement can be made with higher precision and sensitivity when the electrode with micro pillars is used, than when the electrode without micro pillars is used.

This can be explained as follows: Formation of micro pillars on the surface of the electrode provides a drastic increase in the surface area of the electrode substrate in the present invention over the smooth electrode (electrode without micro pillars), with the result that there is an increase in the amount of copper deposited on the surface of the electrode.

TABLE 1
Measurements by anode stripping method

| | Electric charge Q₁ of high- |
|---|---|
| | sensitive electrode for dissolving |
| Copper ion | copper/Electric charge Q₂ of a the |
| concentration | smooth electrode for dissolving copper |
| 1 × 10⁻⁸ mol/l | 10.5 |
| 1 × 10⁻⁹ mol/l | 11.0 |
| 1 × 10⁻¹⁰ mol/l | 13.5 |
| 1 × 10⁻¹¹ mol/l | 14.8 |

The following describes the other specific embodiments of the present invention:

(1) An optical device according to any one of the claims characterized in that a group of columnar micro pillars mainly composed of organic substances is arranged according to a predetermined arrangement method, and a photonic band gap and optical path are formed to allow input and output of optical signals.

(2) An optical device according to any one of the claims characterized in that a group of columnar micro pillars composed of two opposing substrates and the group of columnar micro pillars consisting of organic polymer in the space sandwiched between these substrates is arranged according to a predetermined arrangement method, and an optional path is constituted by formation of a photonic band gap and a specific area devoid of the aforementioned group of columnar micro pillars.

(3) An optical device according to any one of the claims characterized in that the aforementioned group of columnar micro pillars is connected to a thin film formed on the surface of the substrate.

(4) An optical signal processing device according to any one of the claims characterized in that the aforementioned organic polymer contains at least one type of substance selected from the group consisting of particles of oxide, nitride and metal.

(5) An optical signal processing device according to any one of the claims further comprising an optical signal input device and a light receiving device.

(6) A functioning substrate according to any one of the claims further containing a group of columnar micro pillars characterized in that the aforementioned micro pillar and the substrate connected with this micro pillar are arranged integrally with each other.

(7) A functioning substrate according to any one of the claims further having a group of columnar micro pillars characterized in that the aforementioned organic material is mainly composed of a thermoplastic high molecular material.

(8) A functioning substrate according to any one of the claims further containing a group of columnar micro pillars characterized in that the aforementioned organic material is a photo-cured high molecular material.

(9) A functioning substrate according to any one of the claims further containing an group of columnar micro pillars characterized in that the main component of the aforementioned group of columnar micro pillars contains at least one type of substance selected from the group consisting of cycloolefin polymer, polymethyl methacrylate, polystyrene, polycarbonate, polyethylene terephthalate, polylactic acid and polypropylene.

(10) A functioning substrate according to any one of the claims further having a group of columnar micro pillars characterized in that the main component of the aforementioned group of columnar micro pillars has a second matrix on the aforementioned first matrix through a spacer.

(11) An optical circuit wherein:
an optical connector and a plurality of optical output units optically connected with this connector are mounted on one and the same substrate;
at least one of the aforementioned optical connector and optical output unit has a first matrix of organic material and a group of columnar micro pillars of organic material extending from this matrix;
the tip end of this micro pillar group contacts the second matrix;
this micro pillar has an equivalent diameter from 10 nm through 10 μm and a height of 50 nm through 10 μm;
the aspect ratio of this micro pillar is not less than 4, and
this micro pillar group is arranged to constitute at least one optical path;
the aforementioned optical device having one or more light input sections and one or more light output sections.

(12) An optical signal processing apparatus, wherein:
a first polymer surface and a group of columnar micro pillars of organic polymer extending from the first organic polymer surface are provided;
the tip end of the group of columnar micro pillars contacts the second organic polymer surface;
the micro pillar has an equivalent diameter of 10 nm to 10 μm and a height of 50 nm through 10 μm;
the aspect ratio of the columnar micro pillar is not less than 4,
the group of columnar micro pillars is arranged to constitute at least one optical path; and
two or more optical devices each having one or more light input sections and one or more light output sections are optically coupled with one another.

(13) A functioning substrate having a group of micro pillars made of organic polymer, wherein the micro pillars are self-supporting and arranged on a base member supporting the micro pillars, and wherein each of the micro pillars has an aspect ratio of 4 or larger, a diameter of 1 μm or less, and a height of 100 μm or less.

(14) The functioning substrate according to any one of claims, wherein the equivalent diameter of one end of the micro pillars is smaller than that of the other end of the micro pillars, the ends of the micro pillars that have the smaller diameter being connected to the supporting member.

(15) The functioning substrate according to any one of claims, wherein a plurality of layers of the micro pillars are supported on the supporting member, each of the layers being bonded to supporting members.

1. A cell culture sheet comprising:
a matrix having at least one group of columnar micro pillars extending from the matrix, said at least one group of columnar micro pillars being formed by pressing a mold, having pits, against the matrix such that material of the columnar micro pillars is pressed into the pits, and separating the mold therefrom;
wherein said at least one group of columnar micro pillars is adapted to have a cell thereon for culturing thereof.

2. The cell culture sheet according to claim 1, wherein said columnar micro pillars are made of an organic polymer material.

3. The cell culture sheet according to claim 2, wherein said matrix includes a film of said organic polymer material.

4. The cell culture sheet according to claim 1, having a least two groups of columnar micro pillars, and wherein a gap is provided between two groups of said at least two groups of columnar micro pillars.

5. The cell culture sheet according to claim 4, wherein said gap is formed by removing some of said columnar micro pillars.

6. The cell culture sheet according to claim 1, wherein an aspect ratio of equivalent diameter (D) to height (H) of each of said columnar micro pillars is at least 4.

7. The cell culture sheet according to claim 1, wherein said at least one group of columnar micro pillars is also adapted to have culturing media and culture solution provided thereon together with said cell.

8. The cell culture sheet according to claim 1, wherein the columnar micro pillars are integral with material of the matrix.

9. The cell culture sheet according to claim 1, wherein said at least one group of columnar micro pillars is formed by elongating material of the columnar micro pillars upon separating the mold from the material of the columnar micro pillars pressed into the pits.

10. The cell culture sheet according to claim 1, wherein said at least one group of columnar micro pillars is formed by separating said mold from said material and stretching part of said material in said pits.

11. Culturing structure comprising the cell culture sheet according to claim 1 in a vessel for culturing said cell.

12. Culturing structure according to claim 11, wherein said vessel also includes a culture solution.

13. Culturing structure according to claim 11, wherein said vessel is a glass-made Petri dish.
